(19) United States
(12) Patent Application Publication
(10) Pub. No.: US 2024/0102116 A1
(43) Pub. Date: Mar. 28, 2024

(54) A PROCESS AND A KIT FOR DETECTION OF CORONAVIRUS AND OTHER RESPIRATORY VIRUSES

(71) Applicant: Council of Scientific and Industrial Research, New Delhi (IN)

(72) Inventors: Sai Uday Kiran Peddapuvala, Hyderabad (IN); Gokulan Coimbatore Gurumoorthy, Hyderabad (IN); Santosh Kumar Kuncha, Hyderabad (IN); Karthik Bharadwaj Tallapaka, Hyderabad (IN); Rakesh Kumar Mishra, Hyderabad (IN)

(21) Appl. No.: 18/255,748

(22) PCT Filed: Mar. 3, 2021
(86) PCT No.: PCT/IN2021/050198

§ 371 Date: Jun. 2, 2023

(30) Foreign Application Priority Data
Dec. 4, 2020 (IN) 202011053106

(51) Int. Cl. C12Q 1/70

(52) U.S. Cl. CPC C12Q 1/701

(57) ABSTRACT

The present invention is directed towards a process of RT-PCR based diagnosis of SARS-CoV2 and other related respiratory viruses without storing and transporting the naso-pharyngeal and oro-pharyngeal swabs in Viral Testing Media directly after extracting the viral particles from the swabs in a suitable buffer, and using these extracts directly, without involving any steps of RNA isolation/extraction, for RT-PCR based diagnosis of SARS-CoV2. The present invention is also directed towards kits for time and cost-efficient diagnosis of SARS-CoV2 and other related respiratory viruses at efficacies matching or better than the efficacies of the gold-standard RT-PCR method starting from VTM transported swabs for diagnosing the same. The present invention also directs to variant process wherein the viral particles eluted in suitable buffers are further subjected to the conventional steps of RNA extraction before qRT-PCR.

### FIELD OF THE INVENTION

The present invention relates to a novel process and kit for detection of novel coronavirus and other respiratory viruses. In particular, the present invention relates to a new process in the field of biotechnology for the detection of viral diseases directly from dry nasopharyngeal and oropharyngeal swabs without the need of storing and transporting them in Virus Transport Medium (VTM). This process provides a better solution to elute the virus particles directly in a buffer, inactivate, and directly performing qRT-PCR without the steps of RNA extraction from the collected virus swab samples. Another variant of this process involves a combination of dry swab and RNA extraction before performing qRT-PCR and leads to an enhanced efficacy of the process of diagnosis of SARS-CoV-2. Due to its low cost, wherein the existing cost per test can be brought down by 40-50% with the developed invention and time effective nature, the instant invention has the potential to replace the existing standard methods of SARS-CoV-2 diagnosis and can be very well extended to the diagnosis of other respiratory infections. Indirectly; this invention reduces the usage of plastic wares that are otherwise used for RNA extraction in the standard method and thus saves the environment.

### BACKGROUND OF THE INVENTION AND DESCRIPTION OF PRIOR ART

Efficient diagnosis of an infectious pandemic carries inherent challenges such as biosafety during sample handling, skilled manpower, time consumption for testing, sensitivity of the testing method and significant economic burden, irrespective of the nations. The present SARS-CoV-2 is no exception for this, where the most efficient/reliable screening method is Reverse Transcription-Polymerase Chain Reaction (RT-PCR)-based detection of viral nucleic acid from the patient sample (Al-Tawfiq and Memish, 2020; Emery et al., 2004). Rapidly growing number of coronavirus disease 2019 (COVID-19) cases warrants reliable and quicker testing methods (Wu et al., 2020). In the absence of specific drug and/or vaccine, the only way to control SARS-CoV-2 spread is large scale screening and isolation of the infected individuals at early stages of infection. Screening using antibody-based methods is rapid but cannot be used for early stage detection (Carter et al., 2020). Despite being a superior method, RT-PCR demands significant amount of time due to a laborious and expensive RNA isolation step from the Viral Transport Medium (VTM) containing the swab samples. Currently, the challenge is to adapt a detection method which is quicker and still retaining the sensitivity of the standard RT-PCR-based method. Different studies have previously reported inexpensive, nucleic-acid extraction-free methods for PCR-based clinical diagnosis (Freeman et al., 2018; Simon et al., n.d.).

The commonly known and widely accepted processes of SARS-CoV-2 diagnosis include the collection of swab samples from the nasopharyngeal and/or oropharyngeal cavities and transporting them to the testing centres in a liquid medium called viral transport medium (VTM). VTM helps in maintaining the cells and viral particles (if any) intact and facilitates long term storage of the specimen. In a testing centre, a part of the swab-containing VTM is processed to separate and purify RNA, a biomolecule and a genetic material of certain viruses including SARS-CoV-2. This step is called RNA extraction and is one of the time-consuming and expensive steps in the whole process. In addition, using VTM poses a risk of leakage which could in turn have effects on the healthcare workers handling the same.

Reference may be made to Uday Kiran et al., 2020 which reported that the dry swabs could directly be eluted in Tris-EDTA (TE) buffer and these eluates could be compatible with qRT-PCR without RNA isolation. A similar hypothesis was also tested by other researchers and they also seemed to be of the view that eluting the dry swab with TE buffer alone could support molecular detection of SARS-CoV-2 via end point qRT-PCR without substantially compromising sensitivity (Sanjay Srivatsan et al., 2020). While the current process of eluting the contents of the dry swab seemed to be different from Sanjay Srivatsan et al., 2020, both involved elution in TE buffer, albeit in different volumes, and subjected to different treatments and conditions. However, subsequent validations to test the robustness of this process of directly eluting the contents of the dry swab in a larger sample size, and by varying other parameters, like samples collected from different locations or using different RT-PCR reagents and primers supplied with multiple FDA approved RT-PCR kits for COVID19 detection were not consistent and not so promising. It is pertinent to mention here that Sanjay Srivatsan et al., 2020 had tested only 11 patient samples and their observation was based on only these 11 samples. They also tried a combination of TE buffer and triton-X detergent for eluting the contents of the dry swab, but it simply did not work for them under their experimental conditions. They also seemed to be apprehensive that their hypothesis would need further validations with larger sizes and with multiple varying parameters to further validate this process and the reproducibility and consistency of their initial observations.

Thus, with subsequent experimentations and not so promising results during further validations, rather the dissuadingly negative and/or inconsistent results obtained as compared to the gold standard method; the inventors of the present invention realized that while the viral contents could be extracted in TE buffer, and RT-PCR could be done directly from that extract, but this method hypothesized by the inventors of the instant invention as well as the method proposed by Sanjay Srivatsan et al. had a significant drawback that despite the ease of transportation, and saving substantial amount of time by eliminating the RNA extraction step, it did not match the sensitivity and reproducibility of the Gold Standard method which involved transportation in VTM and conventional RNA extraction before qRT-PCR, and TE buffer extraction alone could not serve as a replacement to the conventional Gold Standard Method to increase the efficacy of the Covid19 testing methods by any means.

Accordingly, keeping in view the drawbacks of the hitherto reported prior arts, the inventors of the present invention realized that there exists a dire need to provide a dry swab process for COVID detection that aims to eliminate the need to collect the samples in VTM as well as the RNA extraction step, which is safe and efficient because of the use of proteinase K in the buffer while ensuring specificity and sensitivity with a large number of samples, wherein the samples are heated before RT-PCR so as to ensure the inactivation of virus thereby making it safer to handle and is cost-effective such that it can be very well adopted by developing nations and if done, it can drastically improve their healthcare systems by helping them allocate more budget for treatment than for diagnosis.

### OBJECTIVES OF THE INVENTION

The major objective of the present invention is therefore to provide a novel process for the diagnosis of SARS-CoV2 and other influenza viruses wherein the nasopharyngeal and/or oropharyngeal swab sample need to be collected for diagnosis without having the requirement of storing and transporting the nasopharyngeal and/or oropharyngeal swab samples in Viral transport Medium (VTM) or any other liquid medium to avoid the hazard that could be caused by the spillage of liquid media during transportation of the swabs from the collection centres to the diagnostic centres.

Another objective of the present invention is to provide a cost-efficient process for the diagnosis of SARS-CoV2 and other viruses in order to make these process and diagnostic kits based on these process accessible to the masses.

Still another objective of the invention is to provide newer process and diagnostic kits for the diagnosis of SARS-CoV2 with increased efficacy of the diagnostic process and diagnostic kits for better diagnosis and subsequent management of the dreaded disease.

Yet another objective of the invention is to provide newer process and develop diagnostic kits which could diagnose SARS-CoV2 viruses quicker than the conventional gold standard method for the diagnosis of SARS-CoV2 by incorporating Proteinase-K.

Still another objective of the invention is to provide newer process and diagnostic kits for diagnosing SARS-CoV2 and other respiratory viruses which are cost and time-efficient without compromising with the efficiency of the diagnostic process.

### SUMMARY OF THE INVENTION

The present invention provides a highly time-efficient and cost-efficient process of diagnosing SARS-CoV2 using dry naso-pharyngeal and oro-pharyngeal swabs while matching the process efficacy of the reported gold standard method of qRT-PCR based diagnosis without RNA isolation and/or extraction.

In one aspect of the present invention, the present disclosure provides a process for detection of SARS-CoV2 and other viruses in a sample. The process comprises collecting, storing and transporting the sample in single-layer packing in a dry form and free from viral transport media (VTM) and any other liquid media from a collection centre to a testing centre; extracting the sample with 250 to 500 μl of a buffer comprising Tris-EDTA buffer of pH in the range of 7.2 to 7.6 and serine protease; heat-inactivating the extracted sample obtained for 3 to 10 minutes at a temperature ranging from 90 to 100 degrees C.; performing direct qRT-PCR from the sample as obtained as per the RT-PCR kit manufacturer's instructions manual; and interpreting the qRT-PCR results as positive, negative or inconclusive as per the RT-PCR kit recommendations to detect the presence of SARS-CoV2.

In another aspect of the present invention, the concentration of Tris in TE buffer is 10 mM, EDTA is 0.1 mM and the Serine protease is in the range of 0.5 to 5.0 mg/ml in the process.

In another aspect of the present invention, wherein the serine protease is Proteinase K.

In another aspect of the present invention, the process further comprises: collecting, storing and transporting the sample in single-layer packing in dry form and free from Viral transport Media (VTM) and any other liquid media from the collection centre to the testing centre; extracting the sample with 250 to 500 μl of TE-Proteinase K buffer (pH-7.4) kit; heat-inactivating the extracted sample for 3 to 10 minutes at a temperature ranging from 90 to 100 degrees; isolating RNA from the heat inactivated TE-proteinase K buffer extract; performing qRT-PCR from the isolated RNA as template; and interpreting the qRT-PCR results as positive, negative or inconclusive as per the RT-PCR kit recommendations to detect the presence of SARS-CoV2.

In another aspect of the present invention, the present invention provides that extraction of the virus particles is carried out in TE-proteinase K buffer comprising 10 mM Tris pH-7.4, 0.1 mM EDTA and 2 mg/ml Proteinase K.

In another aspect of the present invention, the present invention provides a kit for detection of SARS-CoV2 and other viruses in a sample. The kit comprises a collection tube and swabs in first part, TE-proteinase K buffer comprising 10 mM Tris pH-7.4, 0.1 mM EDTA+2 mg/ml Proteinase K, RT-PCR Buffer for extracting the viral particles from the dry swab in second part, and DNA polymerase enzyme, reverse transcriptase enzyme, in-vitro transcribed RNA for RdRp gene, E gene, N gene, ORF1ab gene from SARS-CoV2, primers and probe mix for RdRp gene, primers and probe mix for E gene, primers and probe mix for N gene, primers and probe mix for ORFlab gene, FAM dye, ROX dye, JOE dye and Cy5 dye for RT-PCR in the third part of the kit.

In another aspect of the present invention, the present invention provides a kit wherein the primers, probes, dyes, enzymes and RT-PCR buffer are from FDA approved RT-PCR external manufacturers.

In another aspect of the present invention, the present invention provides a kit for the detection of SARS-CoV2 and other viruses in a sample. The said kit comprises a collection tube and swabs in first part, TE-proteinase K buffer comprising 10 mM Tris pH-7.4, 0.1 mM EDTA+2 mg/ml Proteinase K, RT-PCR Buffer for extracting the viral particles from the dry swab in second part, lysis buffer, spin columns, wash buffers and elution buffer for RNA extraction in third part, and DNA polymerase enzyme, reverse transcriptase enzyme, in-vitro transcribed RNA for RdRp gene, E gene, N gene, ORF1ab gene from SARS-CoV2, primers and probe mix for RdRp gene, primers and probe mix for E gene, primers and probe mix for N gene, primers and probe mix for ORF lab gene, FAM dye, ROX dye, JOE dye and Cy5 dye for RT-PCR in the fourth part of complete kit.

In another aspect of the present invention, the present invention provides use of the kits for in-vitro detection of SARS-CoV2 virus and other related respiratory and influenza viruses.

In another aspect of the present invention, the present invention provides an in-vitro process for detection of SARS-CoV2 and other viruses in a sample. The process comprising: collecting, storing and transporting the sample in a single-layer packing in dry form and free from Viral transport Media (VTM) and any other liquid media from the collection centre to the testing centre; extracting the sample of step [a] with 250 to 500 μl of a buffer comprising Tris-EDTA buffer of pH in the range of 7.2 to 7.6 and serine protease; heat-inactivating the extracted sample obtained for 3 to 10 minutes at a temperature ranging from 90 to 100 degrees C.; performing direct qRT-PCR from the sample as per the RT-PCR kit manufacturer's instructions manual; interpreting the qRT-PCR results as positive, negative or inconclusive as per the RT-PCR kit recommendations.

In another aspect of the present invention, the present invention provides an in-vitro process. The process further comprising: collecting, storing and transporting the sample in single-layer packing in dry form and free from Viral transport Media (VTM) and any other liquid media from the collection centre to the testing centre; extracting the sample with 250 to 500 μl of TE-Proteinase K buffer (pH-7.4) kit; heat-inactivating the extracted sample for 3 to 10 minutes at a temperature ranging from 90 to 100 degrees; isolating RNA from the heat inactivated TE-proteinase K buffer extract obtained; performing qRT-PCR from the isolated RNA obtained as template; interpreting the qRT-PCR results as positive, negative or inconclusive as per the RT-PCR kit recommendations.

In an embodiment, the present invention is directed towards collecting, handling and transporting nasopharyngeal and/or oropharyngeal swab samples in dry form without having the need to store and transport them in any Viral Transport Media for diagnosis of SARS-CoV2 without extracting RNA from the virus.

In another embodiment, the present invention provides a process of diagnosis of SARS-CoV2 and other respiratory viruses by eluting the dry swabs containing viral particles in Tris-EDTA buffer in the presence of serine proteases and directly using the viral eluates after inactivation for qRT-PCR.

In still another embodiment, the present invention provides a process of diagnosis of SARS-CoV2 and other related respiratory viruses by eluting the dry swabs containing viral particles in Tris-EDTA buffer in the presence of proteinase K and directly using the viral eluates for qRT-PCR.

In yet another embodiment, the present invention provides a variant process of the dry-swab method, wherein RNA is extracted from the nasopharyngeal and oropharyngeal swab after eluting the viral particles of the swabs in TE buffer in the presence of serine proteases of the before doing qRT-PCR for diagnosis of SARS-CoV2 and other related respiratory viruses.

In still another embodiment, the present invention provides a variant process of the dry-swab method, wherein RNA is extracted from the TE-Proteinase K eluate of dry nasopharyngeal and oropharyngeal swab before doing qRT-PCR for diagnosis of SARS-CoV2 and other related respiratory viruses.

In yet another embodiment, the present invention provides a variant process of the dry-swab method, wherein RNA is extracted from the TE-proteinase K eluate of dry nasopharyngeal and oropharyngeal swab before doing qRT-PCR with more than 100% increased process efficacy compared to the gold-standard method of VTM and RNA extraction-based diagnosis of SARS-CoV2 and other related respiratory viruses.

In still another embodiment, the present invention provides a kit for the detection of SARS-CoV2 virus from dry nasopharyngeal and oropharyngeal swabs from patients without involving the steps of RNA extraction before qRT-PCR.

In yet another embodiment, the present invention provides a kit for the detection of SARS-CoV2 virus from dry nasopharyngeal and oropharyngeal swabs from patients wherein the buffer for eluting viral particles of the dry swabs contains TE buffer and proteinase K and does not involve the steps of RNA extraction before qRT-PCR.

In still another embodiment, the present invention provides the use of the kit for the detection of SARS-CoV2 virus from dry nasopharyngeal and oropharyngeal swabs from patients without involving the steps of RNA extraction before qRT-PCR.

In yet another embodiment, the present invention provides the use of the kit for the detection of SARS-CoV2 virus from dry nasopharyngeal and oropharyngeal swabs from patients wherein the buffer for eluting viral particles of the dry swabs contains TE buffer and proteinase K and does not involve without involving the steps of RNA extraction before qRT-PCR.

In a further embodiment, the present invention provides a kit for the detection of SARS-CoV2 and other related respiratory viruses from dry nasopharyngeal and oropharyngeal swabs, wherein RNA is extracted from the TE-proteinase K eluate of dry nasopharyngeal and oropharyngeal swab before doing qRT-PCR.

In still further embodiment, the present invention provides the use of the kit for the detection of SARS-CoV2 and other related respiratory viruses from dry nasopharyngeal and oropharyngeal swabs, wherein RNA is extracted from the TE-proteinase K eluate of dry nasopharyngeal and oropharyngeal swab before doing qRT-PCR.

### BRIEF DESCRIPTION OF THE ACCOMPANYING DRAWINGS

FIG. 1 is a schematic representation to explain the Dry swab process for SARS-CoV2 virus diagnosis invented in the present invention. As a first step of this schematic representation, dry swab is transferred to vial containing 400 ul of TE-proteinase K buffer. The next is an incubation of the dry swab in this buffer for 30 minutes. 50 μl of this buffer from step 2 is transferred to a new vial/96 well plate and heated at 98° C. for 6 minutes. This buffer extract is used as the template for carrying out qRT-PCR, while storing the rest of the extract at −80° C. for further usage.

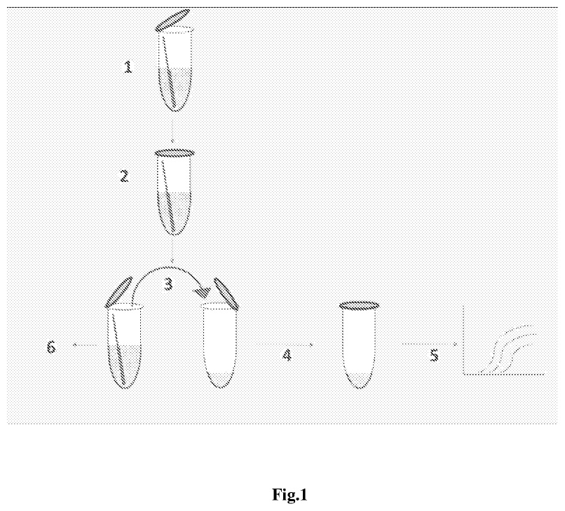

FIG. 2 represents the experimental data comparing the outputs after directly using the VTM extract as a template for RT-PCR versus extracting RNA from the VTM containing swab and using this RNA as a template for RT-PCR (the gold standard method). It is evident from FIG. 2 and also from example 2 that the direct VTM extract does not even remotely match the efficiency of the Gold standard method which involves RNA extraction from VTM extract and then using that RNA as a template for RT-PCR for SARS-CoV2 diagnosis.

FIG. 3A represents the E gene C_{T} values obtained from VTM-RNA (circles) and buffer extract (triangles). It graphically represents the E gene C_{T} values obtained from VTM-RNA (squares) and dry swab process (triangles) (refer table 2). For a majority of the samples the C_{T} values from both the process are comparable, indicating the similar efficiency of the methods. In certain samples, the target was detected in either of the methods.

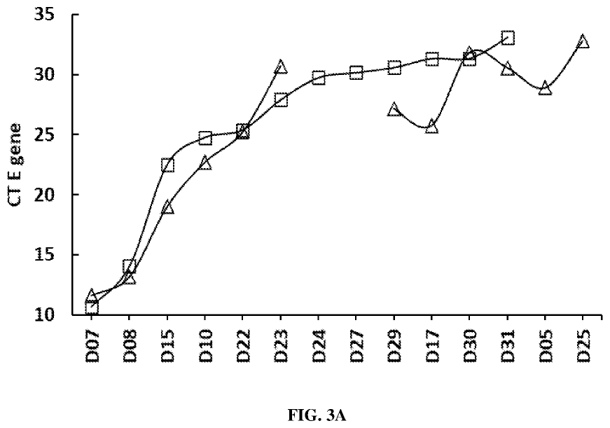

FIG. 3B represents the N gene C_{T} values obtained from VTM-RNA (circles) and buffer extract (triangles). It graphically represents the N gene C_{T} values obtained from VTM-RNA (squares) and dry swab process (triangles) (refer table 2). For a majority of the samples the C_{T} values from both the process are comparable, indicating the similar efficiency of the methods. In certain samples, the target was detected in either of the methods.

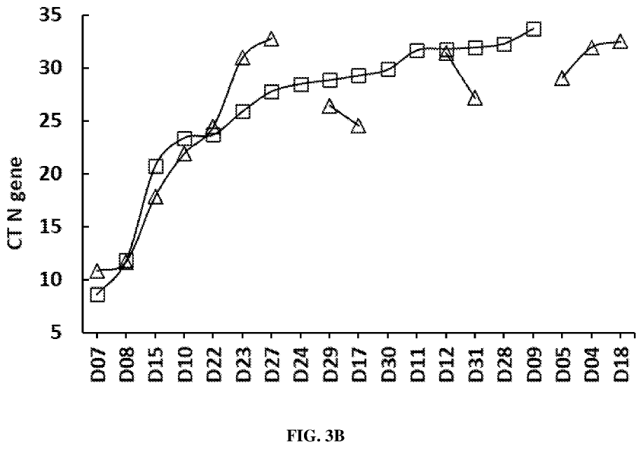

FIG. 3C represents the ORF lab gene C_{T} values obtained from VTM-RNA (squares) and buffer extract (triangles). It graphically represents the ORF lab C_{T} values obtained from VTM-RNA (circles) and dry swab process (triangles) (refer table 2). For a majority of the samples the C_{T} values from both the process are comparable, indicating the similar efficiency of the methods. In certain samples, the target was detected in either of the methods.

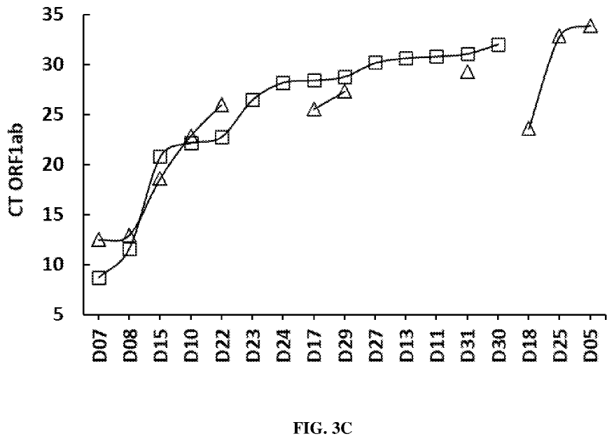

### DETAILED DESCRIPTION OF THE INVENTION

The embodiments are described in sufficient detail to enable those skilled in the art to practice the embodiments and it is to be understood that the logical, mechanical and other changes may be made without departing from the scope of the embodiments. The 15 following detailed description is therefore not to be taken in a limiting sense.

The present invention is directed towards novel processes and kits for the detection of SARS-Cov2 and other related respiratory viruses. The major objective of the invention is to offer a new, safe, cost and time effective process for the diagnosis of SARS-CoV2 and other influenza viruses wherein the nasopharyngeal and/or oropharyngeal swab sample needs to be collected for diagnosis without having the requirement of storing and transporting the nasopharyngeal and/or oropharyngeal swab samples in Viral transport Medium (VTM) or any other liquid medium to avoid the hazard that could be caused by the spillage of liquid media during transportation of the swabs from the collection centres to the diagnostic centres. The detailed steps involved in the process of the present invention are outlined here as under:

### Sample Collection and Transport

The swab samples were collected from voluntary patients at Gandhi Medical College & Hospital, Secunderabad, India. Two nasopharyngeal swabs were collected from each patient and one was transported as dry swab and another in 1 mL VTM (HiMedia Labs, Mumbai) respectively and the samples were kept at 4° C. till further processing.

### Sample Processing

### Complete Sample Processing was Done in the BSL-3 Facility of CSIR-CCMB by Following Standard Operating Procedures—

1. Resuspension/Extraction of Biological Material from Dry Swabs:

The dry swabs were transferred to 1.5 mL microfuge tubes containing 400 μl of TE+Proteinase K buffer [10 mM Tris pH-7.4, 0.1 mM EDTA, 0.5-5 mg/ml Proteinase K], The swabs were cut to make them fit into the tubes and incubated at room temperature for 30 min to ensure the release of biological material.

2. Heat Inactivation:

For direct VTM to RT-PCR, an aliquot of 1 mL VTM samples was diluted 3 times before processing (as the existing recommendation suggests using 3 mL VTM for sample collection). 50 μl of the VTM (for direct VTM to RT-PCR) and TE+Proteinase K extract were aliquoted from the respective vials containing swabs in to separate vials and heated at 90-100° C. for 5-10 minutes on a dry heat block. The inactivated samples were directly used as a template for RT-PCR.

### RNA Isolation

The RNA isolation from 3 mL VTM and TE+Proteinase K buffer (containing dry swab) was performed using the QIAamp Viral RNA isolation kit (Qiagen, Germany) according the manufacturer's protocol. In both cases, 150 μl of the sample was processed for RNA isolation.

### RT-PCR

All the RT-PCR work was carried out in a BSL-2 facility of CSIR-CCMB, Hyderabad, India.

Heat inactivated VTM (direct VTM), TE+Proteinase K buffer extract, and RNA isolated from TE+Proteinase K buffer extract (TE-RNA) and VTM (VTM-RNA) from appropriate samples were tested using the FDA approved FOSUN COVID-19 detection RT-PCR kit (Fosun Pharma USA Inc.). The primer-probe mix targets Envelope gene (E gene; ROX-labelled), Nucleocapsid gene (N gene; JOE-labelled) and Open Reading Frame lab (ORF1ab; FAM-labelled) in the viral genome. The RT-PCR was performed according to the manufacturer protocol. The reactions were multiplexed after performing an in-house standardization (Supplementary Table 1). RT-PCR was performed using QuantStudio 5 (Applied Biosystems, USA).

The buffer extract was directly used as template for the subsequent qRT-PCR reaction with a commercial kit. The reaction conditions were followed as per manufacturer's recommendations. The results were interpreted based on the amplification plot and C_{T} values obtained. FOSUN kit that recommends a C_{T} values cut-off to be less than or equal to 36 for the purposes of data table were used and examples are presented here for exemplary purposes. A sample would be interpreted positive for SARS-CoV-2 if at least two of the three targets are amplified.

C_{T} value is defined as and/or corresponds to the threshold cycle when the amplification of the target is differentiable from the background and it provides a measure of the target abundance. Lesser the value more abundant is the target. Microsoft Excel and Origin software were used to generate the plots.

It was first hypothesized that SARS-CoV-2 nucleic acid could be detected directly by using VTM containing swabs of COVID-19 patients. This methodology (direct VTM method) involves the lysis of the virions (in VTM) by heating a 50 μl aliquot of VTM at 98° C. for 6 minutes, followed by using 4 μl of this as a template for subsequent RT-PCR reaction targeting. However, the results showed a 50% reduction in the detection efficiency of positive samples (n=16) compared to the traditional RNA isolation-based method, i.e., the Gold standard Method for SARS-CoV2 detection.

The inventors were really keen on reducing the time and cost involved in the current methods of SARS-CoV2 virus detection to make it reach diverse populations, but omitting the steps of RNA isolation from VTM extracted viruses did not fetch comparable results. Thus, they were intrigued to think about newer solutions which would actually work for the purpose. Of the multiple combinations that were tried, TE buffer at pH 7.4 seemed to work well initially. But as the dependency was on the available FDA approved kits, it was found that while switching to another lot of RT-PCR kit, more of an accidental kind, TE buffer alone was not sufficient to match the efficacy and sensitivity of the Gold standard method while aiming at omitting the step of RNA isolation/extraction which was crucial to reducing the time of diagnosis and cost of diagnosis.

To address the issue of reproducibility of direct extraction in TE buffer alone which was questionable as the RT-PCR kits would change frequently subject to availability and by being manufactured by different manufacturers, a universal solution which would work with the same efficacy and same degrees of reproducibility irrespective of which RT-PCR kits would be used was proposed. With multiple trials and failures, the solution came in the form of TE buffer loaded with proteinase K, which was validated multiple times with different RT-PCR kits, and which has really been working seamlessly and seems a universally acceptable condition (refer Table 1 in examples).

The same set of 33 patient samples was tested by four different methods, i.e.,
1. The Gold Standard Method,
2. Dry Swab method with swab extracted in TE buffer alone,
3. Dry Swab method with swab extracted in TE buffer+Proteinase K,
4. Dry Swab method with swab extracted in TE buffer+Proteinase K followed by RNA extraction.

The results from the above recited 4 methods have been presented in Tables in examples. Of the 33 samples tested using the VTM Gold standard method, 13 samples tested positive and 16 samples tested negative. The report for 4 other samples from these 33 were inconclusive by this method under our experimental conditions.

However, when the same 33 samples were tested using the method 2 above, i.e., dry swab method with swab extracted in TE buffer alone, only 4 samples tested positive and 21 samples tested negative. The report for 8 other samples were inconclusive by this method under our experimental conditions. Hence, it was clear to us that the sensitivity of this process with TE buffer alone did not even nearly match the efficiency of the VTM Gold method of SARS-CoV2 detection.

However, when the same 33 samples were tested using the method 3 above, i.e., dry swab method with swab extracted in TE buffer+Proteinase K, 12 samples tested positive and 17 samples tested negative. The number of inconclusive results was 4 out of the 33 using this process under our experimental conditions. Our results here nearly match the concordance levels of the VTM gold standard method, showing a deviation of only one patient sample from positive to negative in this process and the samples remaining inconclusive by the method invented by us were found inconclusive with the VTM gold standard as well, while comparing the outputs from the Gold standard method vs. this method invented by us, while our invention significantly simplifies the process of detection along with sample collection and is tremendously cost- and time-efficient as well.

Apparently, when the same 33 samples were tested using the process 4 above, i.e., a variant of the dry swab process with swab extracted in TE buffer+Proteinase K wherein RNA was extracted after TE+Proteinase K extraction, and this RNA was used for RT-PCR, 29 samples tested positive and only 3 samples tested negative. The number of inconclusive results here was only 1 out of the 33 using this process comparing 4 by the VTM gold standard method and the dry swab process without RNA extraction invented by us. Our results here showed a concordance of more than 200% compared with the VTM gold standard method, showing an increase in positively tested patient sample from 13 to 29 and there was only one sample (compared to 4 earlier) for which a conclusive test result could not be arrived at using this variant method. Hence the concordance of this variant process increased by at least 100% compared to the Gold standard method of SARS-Cov2 testing.

A sample collection strategy in the form of dry swabs was also introduced, which enhances biosafety during collection, transportation, and processing of samples, as there is no scope of spillage. Also, this method drastically reduces the cost incurred by eliminating VTM and RNA-extraction step. The procedure has been standardized which is now consistent and compelling.

One of the biggest challenges in diagnostics is overcoming the problem of false-negatives, and SARS-CoV-2 is not an exception to this. Recent reports have shown that the percentage of false negatives reported for SARS-CoV-2 is between 20% and 40% with the onset of symptoms and varies with respect to the phase of infection (Kucirka et al., 2020; Li et al., 2020), which is alarming and calls for immediate improvements in the detection methodology. To address this issue, the inventors of the instant invention combined the TE-proteinase K extraction method with traditional method that includes RNA-extraction. Here RNA was first isolated from TE extract+Proteinase K (described earlier in the detailed description), followed by RT-PCR. It was pleasantly surprising that almost half of the total 33 samples which were consistently negative with traditional VTM-based method and also with direct-RT-PCR process turned out to be positive for SARS-CoV-2 when we tried the variant process invented in the present invention (See Table 3). Therefore, it may be well stated that the new hybrid process of TE-Proteinase K based sample extraction, followed by RNA isolation, results in increasing the overall efficiency by almost 100%. These results put forth a remarkable improvement in the detection of SARS-CoV-2 patients, while using less viral load and therefore provides a better opportunity to manage the pandemic. Further, improved detection efficiency provides an avenue for adapting the presently developed process in combination with pooling strategies.

Accordingly, the present invention is directed towards collecting, handling and transporting nasopharyngeal and/or oropharyngeal swab samples in dry form without having the need to store and transport them in any Viral Transport Media for diagnosis of SARS-CoV2 without extracting RNA from the virus.

In an aspect, the present invention provides a highly time-efficient and cost-efficient process of diagnosing SARS-CoV2 using dry naso-pharyngeal and oro-pharyngeal swabs while matching the process efficacy of the gold standard method of qRT-PCR based diagnosis without RNA isolation and/or extraction.

In another aspect, the present invention provides a process of diagnosis of SARS-CoV2 and other related respiratory viruses by eluting the dry swabs containing viral particles in Tris-EDTA buffer and directly using the viral eluates for qRT-PCR.

In another aspect, the present invention provides a process of diagnosis of SARS-CoV2 and other related respiratory viruses by eluting the dry swabs containing viral particles in Tris-EDTA buffer in the presence of serine proteases and directly using the viral eluates for qRT-PCR.

In another aspect, the present invention provides a process of diagnosis of SARS-CoV2 and other related respiratory viruses by eluting the dry swabs containing viral particles in Tris-EDTA buffer in the presence of proteinase K and directly using the viral eluates for qRT-PCR.

In another aspect, the present invention provides a method which uses 250-500 ul of Tris-EDTA buffer with serine protease for extracting the viral particles from the dry swab.

In another aspect, the present invention provides a process wherein total 400 ul of Tris-EDTA buffer with serine protease is used for extracting the viral particles from the dry swab.

In another aspect of the present invention, the pH of the extraction buffer ranges between 7.2 to 7.6.

In another aspect of the present invention, the concentration of serine protease ranges between 0.5 mg/ml to 5 mg/ml.

In another aspect of the present invention, the preferred serine protease is Proteinase K.

In another aspect of the present invention, the most effective concentration of Proteinase K is 2 mg/ml of the TE buffer.

In another aspect of the present invention, the concentration of Tris is 10 mM and the concentration of EDTA is 0.1 mM for the TE buffer.

In another aspect of the present invention, the TE extracted patient sample is heat inactivated for 3-10 minutes at 90-100 degrees C.

In another aspect of the present invention, the heat inactivation of samples is done at 98 degrees C. for 6 minutes.

In another aspect, the present invention is directed towards a variant process of the dry-swab method, wherein RNA is extracted from the TE buffer eluate of dry nasopharyngeal and oropharyngeal swab before doing qRT-PCR for diagnosis of SARS-CoV2 and other related respiratory viruses.

In another aspect, the present invention is directed towards a variant process of the dry-swab method, wherein RNA is extracted from the nasopharyngeal and oropharyngeal swab after eluting the viral particles of the swabs in TE buffer in the presence of serine proteases followed by inactivation of viruses before doing qRT-PCR for diagnosis of SARS-CoV2 and other related respiratory viruses.

In another aspect, the present invention is directed towards a variant process of the dry-swab method, wherein RNA is extracted from the TE-proteinase K eluate of dry nasopharyngeal and oropharyngeal swab before doing qRT-PCR for diagnosis of SARS-CoV2 and other related respiratory viruses.

In another aspect, the present invention is directed towards a variant process of the dry-swab method, wherein RNA is extracted from the TE-proteinase K eluate of dry nasopharyngeal and oropharyngeal swab before doing qRT-PCR with almost 100% increased process efficacy compared to the gold-standard method of VTM and RNA extraction-based diagnosis of SARS-CoV2 and other related respiratory viruses.

In another aspect, the present invention provides a kit for the detection of SARS-CoV2 virus from dry nasopharyngeal and oropharyngeal swabs from patients without involving the steps of RNA extraction before qRT-PCR.

In another aspect, the present invention provides a kit for the detection of SARS-CoV2 virus from dry nasopharyngeal and oropharyngeal swabs from patients wherein the buffer for eluting viral particles of the dry swabs contains TE buffer and proteinase K and does not involve the steps of RNA extraction before qRT-PCR.

In another aspect, the present invention provides the use of the kit for the detection of SARS-CoV2 virus from dry nasopharyngeal and oropharyngeal swabs from patients without involving the steps of RNA extraction before qRT-PCR.

In another aspect, the present invention provides the use of the kit for the detection of SARS-CoV2 virus from dry nasopharyngeal and oropharyngeal swabs from patients wherein the buffer for eluting viral particles of the dry swabs contains TE buffer and proteinase K and does not involve the steps of RNA extraction before qRT-PCR.

In another aspect, the present invention provides a kit for the detection of SARS-CoV2 and other related respiratory viruses from dry nasopharyngeal and oropharyngeal swabs, wherein RNA is extracted from the TE-proteinase K eluate of dry nasopharyngeal and oropharyngeal swab before doing qRT-PCR.

In another aspect, the swabs used in this invention for collecting naso- and oro-pharyngeal samples are nylon swabs.

In another aspect, the present invention provides the use of the kit for the detection of SARS-CoV2 and other related respiratory viruses from dry nasopharyngeal and oropharyngeal swabs, wherein RNA is extracted from the TE-proteinase K eluate of dry nasopharyngeal and oropharyngeal swab before doing qRT-PCR.

In a further aspect, the present invention discloses a novel process of RT-PCR based diagnosis of SARS-CoV2 and other related respiratory viruses without storing and transporting the naso-pharyngeal and oro-pharyngeal swabs in Viral Testing Media directly after extracting the viral particles from the swabs in a suitable buffer, and using these extracts directly, without involving any steps of RNA isolation/extraction in between, for RT-PCR based diagnosis of SARS-CoV2 and other related respiratory viruses.

The present invention also discloses kits based on this novel process for time and cost-efficient diagnosis of SARS-CoV2 and other related respiratory viruses at efficacies matching or better than the efficacies of the gold-standard RT-PCR method starting from VTM transported swabs for diagnosing the same and use thereof.

The present invention also discloses a variant process wherein the viral particles eluted in suitable buffers are further subjected to the conventional steps of RNA extraction before qRT-PCR but with a significant increase in the efficacy of the process compared to the gold-standard method for identification of SARS-CoV2 and other related respiratory viruses.

The present invention also discloses kits based on this variant process for increased efficacy of the diagnostic process compared to kits based on gold-standard RT-PCR method starting from VTM transported swabs for diagnosing the same.

In an aspect, the present invention provides a novel process for the detection of SARS-CoV2 and other viruses in a sample comprising the steps of:
a) collecting, storing and transporting the sample in a single-layer packing in a dry form and free from viral transport media (VTM) and any other liquid media from the collection centre to the testing centre;
b) extracting the sample of step [a] with 250 to 500 μl of a buffer comprising Tris-EDTA buffer of pH in the range of 7.2 to 7.6 and a serine protease;
c) heat-inactivating the extracted sample of step [b] for 3 to 10 minutes at a temperature ranging from 90 to 100 degrees C.;
d) performing direct qRT-PCR from the sample as obtained in step [c] as per the RT-PCR kit manufacturer's instructions manual;
e) interpreting the qRT-PCR results as positive, negative or inconclusive as per the RT-PCR kit recommendations.

In another aspect the present invention provides a process, wherein the concentration of Tris in TE buffer is 10 mM, EDTA is 0.1 mM and the Serine protease is in the range of 0.5 to 5.0 mg/ml.

In still another aspect the present invention provides a process, wherein the serine protease is Proteinase K.

In yet another aspect the present invention provides a process, wherein it further comprises the steps of:
i. collecting, storing and transporting the sample in single-layer packing in dry form and free from Viral transport Media (VTM) and any other liquid media from the collection centre to the testing centre;
ii. extracting the sample of step (i) with 250 to 500 μl of TE-Proteinase K buffer (pH-7.4) kit;
iii. heat-inactivating the extracted sample of step (ii) for 3 to 10 minutes at a temperature ranging from 90 to 100 degrees;
iv. isolating RNA from the heat inactivated TE-proteinase K buffer extract of step (iii);
v. performing qRT-PCR from the isolated RNA of step (iv) as template;
vi. interpreting the qRT-PCR results as positive, negative or inconclusive as per the RT-PCR kit recommendations.

In still another aspect the present invention provides a process, wherein the extraction of the virus particles is carried out in TE-proteinase K buffer comprising 10 mM Tris pH-7.4, 0.1 mM EDTA and 2 mg/ml Proteinase K.

In yet another aspect the present invention provides a kit for the detection of SARS-CoV2 and other viruses in a sample wherein the said kit comprises a collection tube and swabs in first part, TE-proteinase K buffer comprising 10 mM Tris pH-7.4, 0.1 mM EDTA+2 mg/ml Proteinase K, RT-PCR Buffer for extracting the viral particles from the dry swab in second part, and DNA polymerase enzyme, reverse transcriptase enzyme, in-vitro transcribed RNA for RdRp gene, E gene, N gene, ORF1ab gene from SARS-CoV2, primers and probe mix for RdRp gene, primers and probe mix for E gene, primers and probe mix for N gene, primers and probe mix for ORF1ab gene, FAM dye, ROX dye, JOE dye and Cy5 dye for RT-PCR in the third part of the complete kit.

In still another aspect the present invention provides a kit and a process, wherein the primers, probes, dyes, enzymes and RT-PCR buffer are from FDA approved RT-PCR external manufacturers.

In yet another aspect the present invention provides a kit for the detection of SARS-CoV2 and other viruses in a sample wherein the said kit comprises a collection tube and swabs in first part, TE-proteinase K buffer comprising 10 mM Tris pH-7.4, 0.1 mM EDTA+2 mg/ml Proteinase K, RT-PCR Buffer for extracting the viral particles from the dry swab in second part, lysis buffer, spin columns, wash buffers and elution buffer for RNA extraction in third part, and DNA polymerase enzyme, reverse transcriptase enzyme, in-vitro transcribed RNA for RdRp gene, E gene, N gene, ORF1ab gene from SARS-CoV2, primers and probe mix for RdRp gene, primers and probe mix for E gene, primers and probe mix for N gene, primers and probe mix for ORF1ab gene, FAM dye, ROX dye, JOE dye and Cy5 dye for RT-PCR in the fourth part of complete kit.

In still another aspect the present invention provides the use of the developed kits for in-vitro detection of SARS-CoV2 virus and other related respiratory and influenza viruses.

In yet another aspect the present invention provides an in-vitro process for the detection of SARS-CoV2 and other viruses in a sample, the process comprising:
a. collecting, storing and transporting the sample in single-layer packing in a dry form and free from Viral transport Media (VTM) and any other liquid media from a collection centre to a testing centre;
b. extracting the sample of step [a] with 250 to 500 μl of a buffer comprising Tris-EDTA buffer of pH in the range of 7.2 to 7.6 and a serine protease;
c. heat-inactivating the extracted sample of step [b] for 3 to 10 minutes at a temperature ranging from 90 to 100 degrees C.;
d. performing direct qRT-PCR from the sample as obtained in step [c] as per the RT-PCR kit manufacturer's instructions manual;
e. interpreting the qRT-PCR results as positive, negative or inconclusive as per the RT-PCR kit recommendations.

In still another aspect the present invention provides an in-vitro process, wherein the process further comprising:
i. collecting, storing and transporting the sample in single-layer packing in the dry form and free from Viral transport Media (VTM) and any other liquid media from the collection centre to the testing centre;
ii. extracting the sample of step (i) with 250 to 500 μl of TE-Proteinase K buffer (pH-7.4) kit;
iii. heat-inactivating the extracted sample of step (ii) for 3 to 10 minutes at a temperature ranging from 90 to 100 degrees;
iv. isolating RNA from the heat inactivated TE-proteinase K buffer extract of step (iii);
v. performing qRT-PCR from the isolated RNA of step (iv) as template;
vi. interpreting the qRT-PCR results as positive, negative or inconclusive as per the RT-PCR kit recommendations.

### EXAMPLES

The following examples are given by way of illustration of the present invention and therefore should not be construed to limit the scope of the present invention.

The primers, probes, dyes, enzymes and RT-PCR buffer used in the present invention are sourced from FDA approved RT-PCR external manufacturers and lysis buffer, spin columns, wash buffers and elution buffer for RNA extraction are from Qiagen RNA extraction kit.

The enzyme Proteinase-K used for the purposes of the present invention was obtained from M/s G-Biosciences, USA vide Catalogue No—786-043.

### Example 1

### Direct RT-PCR for SARS-CoV2 Virus Diagnosis Using VTM Extract as a Template for RT-PCR

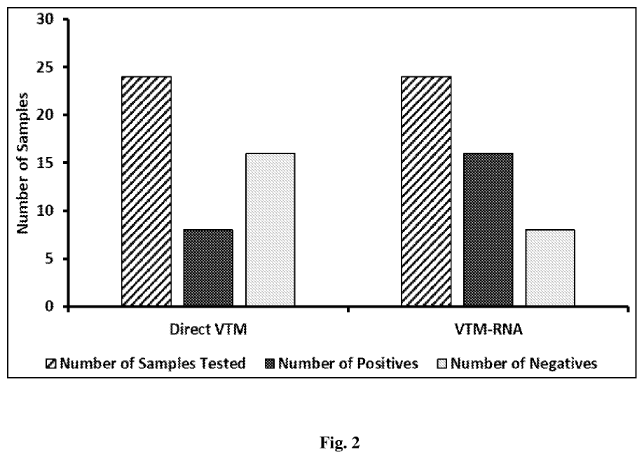

The viral particles present in the dry nasopharyngeal and/or oro-pharyngeal swabs were extracted in 400 μl Viral Transport Media (VTM) and the samples were heat inactivated at 98 degrees C. for 6 minutes. These VTM extracts were directly used as templates for qRT-PCR. The results we got from this RT-PCR were not comparable with the results obtained for the same samples from gold standard method, that is, when RNA was extracted from VTM extract and that RNA was used as a template for qRT-PCR. The same findings have been presented in the form of FIG. 2 in the drawings and figures for this.

### Example 2

Direct RT-PCR from the TE Buffer Extract of Dry Swab for SARS-CoV2 Virus Diagnosis

The viral particles present in the dry nasopharyngeal and/or oro-pharyngeal swabs were extracted in 400 ul TE (pH 7.4) buffer [10 mM Tris pH-7.4, 0.1 mM] and the samples were heat inactivated at 98 degrees C. for 6 minutes. These TE extracts were directly used as templates for qRT-PCR. The results we got from this RT-PCR were initially found similar to the results obtained from the Gold-standard Method. However, when we started adopting this process with TE extraction for screening patient samples in larger sizes, the results obtained using this process were not consistently efficient or equivalent to the results obtained from the Gold standard method.

Table 1 below presents a comparison between the efficacy of the gold standard method of SARS-CoV2 virus detection versus the efficacy of the process of dry swab-based SARS-CoV2 virus detection wherein the viral particles from the dry swab are extracted in TE buffer (pH 7.4) alone and directly subjected to qRT-PCR.

TABLE 1
Comparison between gold standard method of qRT-PCR vs. qRT-PCR from dry swab
extracted in TE buffer alone without RNA extraction for SARS-COV2 detection

| | VTM RNA | | | | Dry swab method with | | | |
|---|---|---|---|---|---|---|---|---|
| | (Gold Standard) | | | | TE buffer extract | | | |
| | E-gene | N-gene | ORF-1AB | | E-gene | N-gene | ORF-1AB | |
| Sample | Ct value | Ct value | Ct value | Interpretation | Ct value | Ct value | Ct value | Interpretation |
| 1 | | | | NEGATIVE | | | | NEGATIVE |
| 2 | | | | NEGATIVE | | | | NEGATIVE |
| 3 | | | | NEGATIVE | | | | NEGATIVE |
| 4 | | | | NEGATIVE | | | 38.48 | INCONCLUSIVE |
| 5 | | | | NEGATIVE | 31.71 | | | INCONCLUSIVE |
| 6 | | | | NEGATIVE | | | | NEGATIVE |
| 7 | 10.72 | 8.62 | 8.73 | POSITIVE | 17.73 | 14.59 | 16.28 | POSITIVE |
| 8 | 14.06 | 11.82 | 11.63 | POSITIVE | 22.16 | 18.66 | 20.96 | POSITIVE |
| 9 | | 33.71 | | INCONCLUSIVE | | 32.64 | | INCONCLUSIVE |
| 10 | 24.77 | 23.39 | 22.19 | POSITIVE | | | | NEGATIVE |
| 11 | | 31.67 | 30.84 | POSITIVE | | | | NEGATIVE |
| 12 | | 31.78 | | INCONCLUSIVE | | | | NEGATIVE |
| 13 | | | 30.68 | INCONCLUSIVE | | | | NEGATIVE |
| 14 | | | | NEGATIVE | | | | NEGATIVE |
| 15 | 22.52 | 20.75 | 20.80 | POSITIVE | | 30.99 | 30.01 | POSITIVE |
| 16 | | | | NEGATIVE | | | | NEGATIVE |
| 17 | 31.33 | 29.29 | 28.45 | POSITIVE | | | | NEGATIVE |
| 18 | | | | NEGATIVE | | | | NEGATIVE |
| 19 | | | | NEGATIVE | | 33.63 | | INCONCLUSIVE |
| 20 | | | | NEGATIVE | | | 14.13 | INCONCLUSIVE |
| 21 | | | | NEGATIVE | | | | NEGATIVE |
| 22 | 25.41 | 23.70 | 22.78 | POSITIVE | | | | NEGATIVE |
| 23 | 27.90 | 25.89 | 26.47 | POSITIVE | | | 33.70 | INCONCLUSIVE |
| 24 | 29.74 | 28.49 | 28.23 | POSITIVE | | | | NEGATIVE |
| 25 | | | | NEGATIVE | | | | NEGATIVE |
| 26 | | | | NEGATIVE | | | | NEGATIVE |
| 27 | 30.16 | 27.78 | 30.20 | POSITIVE | | 30.85 | | INCONCLUSIVE |
| 28 | | 32.30 | | INCONCLUSIVE | | | | NEGATIVE |
| 29 | 30.58 | 28.86 | 28.82 | POSITIVE | 31.23 | | 31.17 | POSITIVE |
| 30 | 31.35 | 29.85 | 32.06 | POSITIVE | | | | NEGATIVE |
| 31 | 33.08 | 31.94 | 31.11 | POSITIVE | | | | NEGATIVE |
| 32 | | | | NEGATIVE | | | | NEGATIVE |
| 33 | | | | NEGATIVE | | | 38.06 | INCONCLUSIVE |
| | | | | POSITIVE- 13 | | | | POSITIVE-4 |
| | | | | NEGATIVE-16 | | | | NEGATIVE-21 |
| | | | | INCONCLUSIVE-4 | | | | INCONCLUSIVE-8 |

It is evident from the comparison above that while the Gold standard method (storage in VTM, followed by RNA extraction and qRT-PCR) fetched 13 positive results and 16 negative results and 4 inconclusive results for SARS-CoV2 virus detection from the 33 samples under observation, the dry swab extracted in TE buffer alone (pH 7.4) and directly subjected to qRT-PCR without involving the steps of RNA extraction) fetched only 4 positive, 21 negative and 8 inconclusive results for the same 33 patient samples. This clearly demonstrates that the dry-swab process when extracted in TE buffer alone was not as sensitive and efficient as the Gold Standard method for SARS-CoV2 detection when we started testing patient samples at large scales and used multiple RT-PCR kits approved by the FDA.

### Example 3

Direct RT-PCR from the TE+Proteinase K Buffer Extract of Dry Swab for SARS-CoV2 Virus Diagnosis

The viral particles present in the dry nasopharyngeal and/or oro-pharyngeal swabs were extracted in 400 ul TE-Proteinase K Buffer (pH 7.4) [10 mM Tris pH-7.4, 0.1 mM EDTA, 2 mg/ml Proteinase K] and the samples were heat inactivated at 98 degrees C. for 6 minutes. These TE-Proteinase K extracts were directly used as templates for qRT-PCR. The results we got from this RT-PCR were found similar to the results obtained from the Gold-standard Method.

Table 2 below presents a comparison between the efficacy of the gold standard method of SARS-CoV2 virus detection versus the efficacy of the process of dry swab-based SARS-CoV2 virus detection wherein the viral particles from the dry swab are extracted in TE+proteinase K buffer (pH 7.4) and directly subjected to qRT-PCR.

TABLE 2
Comparison between gold standard method of qRT-PCR vs. qRT-PCR from dry swab extracted
in TE + Proteinase K buffer without RNA extraction for SARS-COV2 detection

| | | | | | Dry swab method (TE + | | | |
|---|---|---|---|---|---|---|---|---|
| | | | | | Proteinase K buffer- | | | |
| | VTM RNA (Gold Standard) | | | | without RNA extraction) | | | |
| | E-gene | N-gene | ORF-1AB | | E-gene | N-gene | ORF-1AB | |
| Sample | Ct value | Ct value | Ct value | Interpretation | Ct value | Ct value | Ct value | Interpretation |
| 1 | | | | NEGATIVE | | | | NEGATIVE |
| 2 | | | | NEGATIVE | | | | NEGATIVE |
| 3 | | | | NEGATIVE | | | | NEGATIVE |
| 4 | | | | NEGATIVE | | 31.95 | | INCONCLUSIVE |
| 5 | | | | NEGATIVE | 28.89 | 29.08 | 33.89 | POSITIVE |
| 6 | | | | NEGATIVE | | | | NEGATIVE |
| 7 | 10.72 | 8.62 | 8.73 | POSITIVE | 11.62 | 10.83 | 12.48 | POSITIVE |
| 8 | 14.06 | 11.82 | 11.63 | POSITIVE | 13.18 | 11.69 | 12.97 | POSITIVE |
| 9 | | 33.71 | | INCONCLUSIVE | | | | NEGATIVE |
| 10 | 24.77 | 23.39 | 22.19 | POSITIVE | 22.70 | 21.91 | 22.89 | POSITIVE |
| 11 | | 31.67 | 30.84 | POSITIVE | | | | NEGATIVE |
| 12 | | 31.78 | | INCONCLUSIVE | | 31.43 | | INCONCLUSIVE |
| 13 | | | 30.68 | INCONCLUSIVE | | | | NEGATIVE |
| 14 | | | | NEGATIVE | | | | NEGATIVE |
| 15 | 22.52 | 20.75 | 20.80 | POSITIVE | 19.00 | 17.87 | 18.62 | POSITIVE |
| 16 | | | | NEGATIVE | | | | NEGATIVE |
| 17 | 31.33 | 29.29 | 28.45 | POSITIVE | 25.76 | 24.55 | 25.54 | POSITIVE |
| 18 | | | | NEGATIVE | | 32.49 | 23.60 | POSITIVE |
| 19 | | | | NEGATIVE | | | | NEGATIVE |
| 20 | | | | NEGATIVE | | | | NEGATIVE |
| 21 | | | | NEGATIVE | | | | NEGATIVE |
| 22 | 25.41 | 23.70 | 22.78 | POSITIVE | 25.23 | 24.44 | 25.99 | POSITIVE |
| 23 | 27.90 | 25.89 | 26.47 | POSITIVE | 30.70 | 30.96 | | POSITIVE |
| 24 | 29.74 | 28.49 | 28.23 | POSITIVE | | | | NEGATIVE |
| 25 | | | | NEGATIVE | 32.79 | | 32.83 | POSITIVE |
| 26 | | | | NEGATIVE | | | | NEGATIVE |
| 27 | 30.16 | 27.78 | 30.20 | POSITIVE | | 32.79 | | INCONCLUSIVE |
| 28 | | 32.30 | | INCONCLUSIVE | | | | NEGATIVE |
| 29 | 30.58 | 28.86 | 28.82 | POSITIVE | 27.18 | 26.45 | 27.32 | POSITIVE |
| 30 | 31.35 | 29.85 | 32.06 | POSITIVE | 31.81 | | | INCONCLUSIVE |
| 31 | 33.08 | 31.94 | 31.11 | POSITIVE | 30.58 | 27.16 | 29.28 | POSITIVE |
| 32 | | | | NEGATIVE | | | | NEGATIVE |
| 33 | | | | NEGATIVE | | | | NEGATIVE |
| | | | | POSITIVE-13 | | | | POSITIVE-12 |
| | | | | NEGATIVE-16 | | | | NEGATIVE-17 |
| | | | | INCONCLUSIVE-4 | | | | INCONCLUSIVE-4 |

It is evident from the comparison above that while the Gold standard method (storage in VTM, followed by RNA extraction and qRT-PCR) fetched 13 positive results and 16 negative results and 4 inconclusive results for SARS-CoV2 virus detection from the 33 samples under observation, the dry swab process invented by us (viral particles from the dry swab extracted in TE+proteinase K buffer (pH 7.4) and directly subjected to qRT-PCR without involving the steps of RNA extraction) also fetched 12 positive, 17 negative and only 4 inconclusive result for the same 33 patient samples.

This clearly demonstrates that the dry-swab process developed by us is equally sensitive as the Gold Standard method for SARS-CoV2 detection, while it brings with it the ease of storage and transport of the dry swabs directly, plus saves a lot of time by omitting the steps of RNA extraction, saves a lot of cost by avoiding expensive reagents of RNA extraction and the VTM. Hence it is evident that the process invented by us drastically reduces the time taken in detection of SARS-CoV2 virus, which is highly needed and in demand considering the fast and uncontrollable spread of the virus that has been happening globally and unmanageable currently. Hence it can be said without any iota of doubt or disbelief that the process invented by us is highly time- and cost-efficient while matching the detection sensitivity and efficacy of the Gold standard method of SARS-CoV2 detection.

### Example 4

RT-PCR for SARS-CoV2 Virus Diagnosis after RNA Extraction from the TE+Proteinase K Buffer Extract Using the Extracted RNA as a Template for RT-PCR

The viral particles present in the dry nasopharyngeal and/or oro-pharyngeal swabs were extracted in 400 ul TE-Proteinase K Buffer (pH 7.4) [10 mM Tris pH-7.4, 0.1 mM EDTA 0.1 mM, 2 mg/ml Proteinase K] and the samples were heat inactivated at 98 degrees C. for 6 minutes. RNA was extracted from these TE-Proteinase K extracts. The RNA isolation from 3 mL TE+Proteinase K buffer (containing dry swab) was performed using the QIAamp Viral RNA isolation kit (Qiagen, Germany) according the manufacturer's protocol. 150 μl of the sample was processed for RNA isolation.

The extracted RNA was used as a template for qRT-PCR. The results we got from this RT-PCR were at least 75% more efficient compared to the results obtained from the Gold-standard Method based on the process-efficacy.

Table 3 below presents a comparison between the efficacy of the gold standard method of SARS-CoV2 virus detection versus the efficacy of the variant of the dry swab method of SARS-CoV2 virus detection wherein the viral particles from the dry swab are extracted in TE+proteinase K buffer (pH 7.4), followed by RNA extraction prior to qRT-PCR.

TABLE 3
Comparison between gold standard method of qRT-PCR vs. qRT-PCR from dry swab extracted
in TE + Proteinase K buffer followed by RNA extraction for SARS-COV2 detection

| | | | | | Dry swab method with TE + | | | |
|---|---|---|---|---|---|---|---|---|
| | | | | | Proteinase K buffer (followed | | | |
| | VTM RNA (Gold Standard) | | | | by RNA extraction) | | | |
| | E-gene | N-gene | ORF-1AB | | E-gene | N-gene | ORF-1AB | |
| Sample | Ct value | Ct value | Ct value | Interpretation | Ct value | Ct value | Ct value | Interpretation |
| 1 | | | | NEGATIVE | 32.31 | 30.79 | 31.11 | POSITIVE |
| 2 | | | | NEGATIVE | 32.29 | 29.83 | 31.30 | POSITIVE |
| 3 | | | | NEGATIVE | | 31.91 | 31.38 | POSITIVE |
| 4 | | | | NEGATIVE | 32.95 | 31.89 | 31.22 | POSITIVE |
| 5 | | | | NEGATIVE | 28.28 | 26.71 | 26.15 | POSITIVE |
| 6 | | | | NEGATIVE | 31.87 | | 31.02 | POSITIVE |
| 7 | 10.72 | 8.62 | 8.73 | POSITIVE | 8.37 | 6.19 | 5.81 | POSITIVE |
| 8 | 14.06 | 11.82 | 11.63 | POSITIVE | 12.29 | 9.84 | 10.31 | POSITIVE |
| 9 | | 33.71 | | INCONCLUSIVE | 33.12 | 31.94 | 30.92 | POSITIVE |
| 10 | 24.77 | 23.39 | 22.19 | POSITIVE | 22.23 | 20.42 | 19.41 | POSITIVE |
| 11 | | 31.67 | 30.84 | POSITIVE | 31.84 | 29.03 | 29.91 | POSITIVE |
| 12 | | 31.78 | | INCONCLUSIVE | 32.04 | 29.16 | 28.54 | POSITIVE |
| 13 | | | 30.68 | INCONCLUSIVE | | | | NEGATIVE |
| 14 | | | | NEGATIVE | 33.01 | 30.52 | 30.30 | POSITIVE |
| 15 | 22.52 | 20.75 | 20.80 | POSITIVE | 18.30 | 16.52 | 15.87 | POSITIVE |
| 16 | | | | NEGATIVE | 27.10 | 24.77 | 23.81 | POSITIVE |
| 17 | 31.33 | 29.29 | 28.45 | POSITIVE | 24.90 | 23.14 | 22.65 | POSITIVE |
| 18 | | | | NEGATIVE | 30.75 | 30.02 | 29.50 | POSITIVE |
| 19 | | | | NEGATIVE | | 30.24 | 30.07 | POSITIVE |
| 20 | | | | NEGATIVE | 30.86 | 29.68 | 29.90 | POSITIVE |
| 21 | | | | NEGATIVE | | 31.91 | 30.99 | POSITIVE |
| 22 | 25.41 | 23.70 | 22.78 | POSITIVE | 24.64 | 22.62 | 22.28 | POSITIVE |
| 23 | 27.90 | 25.89 | 26.47 | POSITIVE | 27.70 | 25.91 | 25.95 | POSITIVE |
| 24 | 29.74 | 28.49 | 28.23 | POSITIVE | 31.15 | 28.98 | 29.04 | POSITIVE |
| 25 | | | | NEGATIVE | 29.31 | 29.70 | 28.38 | POSITIVE |
| 26 | | | | NEGATIVE | | | | NEGATIVE |
| 27 | 30.16 | 27.78 | 30.20 | POSITIVE | 26.88 | 24.64 | 25.37 | POSITIVE |
| 28 | | 32.30 | | INCONCLUSIVE | | 30.37 | 29.98 | POSITIVE |
| 29 | 30.58 | 28.86 | 28.82 | POSITIVE | 26.75 | 24.78 | 24.03 | POSITIVE |
| 30 | 31.35 | 29.85 | 32.06 | POSITIVE | 28.64 | 25.97 | 26.44 | POSITIVE |
| 31 | 33.08 | 31.94 | 31.11 | POSITIVE | 26.28 | 24.48 | 24.68 | POSITIVE |
| 32 | | | | NEGATIVE | | | | NEGATIVE |
| 33 | | | | NEGATIVE | | | 26.17 | INCONCLUSIVE |
| | | | | POSITIVE-13 | | | | POSITIVE-29 |
| | | | | NEGATIVE-16 | | | | NEGATIVE-3 |
| | | | | INCONCLUSIVE-4 | | | | INCONCLUSIVE-1 |

It is evident from the comparison above that while the Gold standard method (storage in VTM, followed by RNA extraction and qRT-PCR) fetched only 13 positive results and 16 negative results and 4 inconclusive results for SARS-CoV2 virus detection from the 33 samples under observation, the variant process of dry swab method invented by us (viral particles from the dry swab are extracted in TE+proteinase K buffer (pH 7.4), followed by RNA extraction prior to qRT-PCR) also fetched 29 positive, 3 negative and only 1 inconclusive result for the same 33 patient samples. This clearly demonstrates the highly increased efficacy of the detection process using the variant process invented by us, compared to the efficacy of the Gold Standard method of SARS-CoV2 detection.

Also, this invention can be further extended to other viral disease diagnostic processes and development of diagnostic kits thereof.

### Advantages of the Invention

1. A simple and affordable method and kit for the rapid and cost-effective diagnosis of SARS-CoV2 virus.
2. A variant of the simple and affordable method and kit with increased efficacy of SARS-CoV2 diagnosis compared to the gold standard qRT-PCR method. The dry swab-qRT PCR method and its variant method can detect SARS-CoV2.
3. The present invention completely obviates the need of Viral Transport Media (VTM) for storing and transporting the nasopharyngeal and/or oropharyngeal swabs collected from patients to the diagnostic set-ups.
4. The present invention additionally obviates the critical step of RNA extraction from nasopharyngeal and/or oropharyngeal swabs and directly uses the viral particles eluted in TE/Proteinase K Buffer for qRT-PCR based diagnosis of the SARS-CoV2 virus. This method is highly time-efficient and cost-efficient both by obviating the need of VTM and RNA extraction reagents and omitting the conventional step of RNA extraction, while essentially matching the diagnostic efficacy and in cent percent concordance with the conventional gold standard method of SARS-CoV2 diagnosis from swabs transported in VTM followed by RNA extraction followed by qRT-PCR for identification of SARS-CoV2 virus, and constitutes a very simplistic diagnostic kit for the diagnosis of SARS-CoV2 for direct qRT-PCR amplification and detection of SARS-CoV2.
5. A variant of the present invention increases the efficacy of the SARS-CoV2 Diagnostic process almost twice compared to the efficacy of the gold standard qRT-PCR method by extracting RNA from the dry swab by eluting the viral particles in a very simplistic yet highly efficient buffer at pH 7.4, and then using the extracted RNA for qRT-PCR based diagnosis of SARS-CoV2 (Refer Table 3).
6. It would only cost approximately half the current cost of RT-PCR based SARS-CoV-2 diagnostic kit when even VTM alone could be omitted from the process of transportation, and for the purpose of RNA extraction. The cost of diagnostic kit and testing would go down further if the dry swab eluted in TE+Proteinase K buffer would directly be used for qRT-PCR without extracting RNA from the swab-eluate.

### REFERENCES

1. Al-Tawfiq, J. A., and Memish, Z. A. (2020). Diagnosis of SARS-CoV-2 Infection based on C_{T} scan vs. RT-PCR: Reflecting on Experience from MERS-CoV. J Hosp Infect.
2. Emery, S. L., Erdman, D. D., Bowen, M. D., Newton, B. R., Winchell, J. M., Meyer, R. F., Tong, S., Cook, B. T., Holloway, B. P., McCaustland, K. A., et al. (2004). Real-time reverse transcription-polymerase chain reaction assay for SARS-associated coronavirus. Emerg Infect Dis 10, 311-316.
3. Wu, F., Zhao, S., Yu, B., Chen, Y. M., Wang, W., Song, Z. G., Hu, Y., Tao, Z. W., Tian, J. H., Pei, Y. Y., et al. (2020). A new coronavirus associated with human respiratory disease in China. Nature 579, 265-269.
4. Carter, L. J., Garner, L. V., Smoot, J. W., Li, Y., Zhou, Q., Saveson, C. J., Sasso, J. M., Gregg, A. C., Soares, D. J., Beskid, T. R., et al. (2020). Assay Techniques and Test Development for COVID-19 Diagnosis. ACS Cent Sci 6, 591-605
5. Kucirka, L. M., Lauer, S. A., Laeyendecker, O., Boon, D., and Lessler, J. (2020). Variation in False-Negative Rate of Reverse Transcriptase Polymerase Chain Reaction-Based SARS-CoV-2 Tests by Time Since Exposure. Ann Intern Med.
6. Sanjay Srivatsan, Peter D. Han, Katrina van Raay, Caitlin R. Wolf, Denise J. McCulloch, Ashley E. Kim, Elisabeth Brandstetter, Beth Martin, Jase Gehring, Wei Chen, Seattle Flu Study Investigators, Sriram Kosuri, Eric Q. Konnick, Christina M. Lockwood, Mark J. Rieder, Deborah A. Nickerson, Helen Y. Chu, Jay Shendure, Lea M. Starita (2020) Preliminary support for a “dry swab, extraction free” protocol for SARS-CoV-2 testing via RT-qPCR. bioRxiv 2020.04.22.056283; doi: latps://doi.orOlft 110112020.04:22.056283.
7. Uday Kiran, C G Gokulan, Santosh Kumar Kuncha, Dhiviya Vedagiri, Bingi Thrilok Chander, Aedula Vinaya Sekhar, Suchitra Dontamala, Arakatla Lohith Reddy, Karthik Bharadwaj Tallapaka, Rakesh K Mishra, Krishnan Harinivas Harshan, Easing diagnosis and pushing the detection limits of SARS-CoV-2, Biology Methods and Protocols, Volume 5, Issue 1, 2020, bpaa017, https://doi.org/10.1093/biomethods/bpaa017

1-11. (canceled)

12. A process for detecting SARS-CoV2 and other viruses in a sample, the process comprising:
(a) collecting, storing, and transporting the sample from a collection center to a testing center, wherein the sample is in single-layer packing, wherein the sample is in a dry form, and wherein the sample is free from viral transport media and any other liquid media;
(b) extracting the sample of (a) with 250 μL to 500 μL of a buffer comprising Tris-EDTA buffer and from 0.5 mg/mL to 5.0 mg/mL serine protease to obtain an extracted sample, the Tris-EDTA buffer having a Tris concentration of 10 mM, an EDTA concentration of 0.1 mM, and a pH from 7.2 to 7.6;
(c) heat-inactivating the extracted sample of (b) for 3 minutes to 10 minutes at a temperature from 90° C. to 100° C. to obtain a heat-inactivated sample;
(d) performing direct qRT-PCR on the heat-inactivated sample of (c) as per a RT-PCR kit manufacturer's instructions manual to obtain qRT-PCR results; and
(e) interpreting the qRT-PCR results as positive, negative, or inconclusive, as per a RT-PCR kit recommendations.

13. The process of claim 12, wherein the serine protease is proteinase K.

14. The process of claim 12, further comprising:
(i) collecting, storing, and transporting the sample from the collection center to the testing center, wherein the sample is in single-layer packing, the sample is in a dry form, and the sample is free from viral transport media and any other liquid media;
(ii) extracting the sample of (i) with 250 μL to 500 μL of a TE-proteinase K buffer of pH 7.4 to obtain an extracted sample;
(iii) heat-inactivating the extracted sample of (ii) for 3 minutes to 10 minutes at a temperature from 90° C. to 100° C. to obtain a heat inactivated TE-proteinase K buffer extract;
(iv) isolating RNA from the heat inactivated TE-proteinase K buffer extract of (iii) to obtain isolated RNA;
(v) performing qRT-PCR using the isolated RNA of (iv) as template to obtain qRT-PCR results; and
(vi) interpreting the qRT-PCR results as positive, negative, or inconclusive as per the RT-PCR kit recommendations.

15. The process of claim 14, wherein extraction of virus particles is carried out in TE-proteinase K buffer comprising 10 mM Tris of pH 7.4, 0.1 mM EDTA, and 2 mg/mL proteinase K.

16. A kit for detecting SARS-CoV2 and other viruses in a sample, the kit comprising:
a first part comprising a collection tube and swabs;
a second part comprising TE-proteinase K buffer comprising 10 m M Tris of pH 7.4, 0.1 mM EDTA+2 mg/mL proteinase K, and RT-PCR buffer for extracting viral particles from a dry swab; and
a third part comprising DNA polymerase enzyme, reverse transcriptase enzyme, in-vitro transcribed RNA for RdRp gene, E gene, N gene, ORF lab gene from SARS-CoV2, primers and probe mix for RdRp gene, primers and probe mix for E gene, primers and probe mix for N gene, primers and probe mix for ORF lab gene, FAM dye, ROX dye, JOE dye, and Cy5 dye for RT-PCR.

17. The kit of claim 16, wherein the primers, probes, dyes, enzymes, and RT-PCR buffer are from FDA approved RT-PCR external manufacturers.

18. A kit for detecting SARS-CoV2 and other viruses in a sample, the kit comprising:
a first part comprising a collection tube and swabs;
a second part comprising TE-proteinase K buffer comprising 10 mM Tris of pH 7.4, 0.1 mM EDTA+2 mg/mL Proteinase K, and RT-PCR buffer for extracting the viral particles from the dry swab;
a third part comprising lysis buffer, spin columns, wash buffers, and elution buffer for RNA extraction; and
a fourth part comprising DNA polymerase enzyme, reverse transcriptase enzyme, in-vitro transcribed RNA for RdRp gene, E gene, N gene, ORF lab gene from SARS-CoV2, primers and probe mix for RdRp gene, primers and probe mix for E gene, primers and probe mix for N gene, primers and probe mix for ORF lab gene, FAM dye, ROX dye, JOE dye, and Cy5 dye for RT-PCR.

19. The process of claim 12, wherein the process is an in-vitro process.

20. The process of claim 19, further comprising:
(i) collecting, storing, and transporting the sample from the collection center to the testing center, wherein the sample is in single-layer packing, the sample is in a dry form, and the sample is free from viral transport media and any other liquid media;
(ii) extracting the sample of (i) with 250 μL to 500 μL of a TE-Proteinase K buffer of pH 7.4 to obtain an extracted sample;
(iii) heat-inactivating the extracted sample of (ii) for 3 minutes to 10 minutes at a temperature ranging from 90° C. to 100° C. to obtain a heat inactivated TE-proteinase K buffer extract;
(iv) isolating RNA from the heat inactivated TE-proteinase K buffer extract of (iii) to obtain isolated RNA;
(v) performing qRT-PCR using the isolated RNA of (iv) as template to obtain qRT-PCR results; and
(vi) interpreting the qRT-PCR results as positive, negative, or inconclusive as per the RT-PCR kit recommendations.
